(19) United States
(12) Plant Patent Application Publication
(10) Pub. No.: US 2003/0172421 P1
(43) Pub. Date: Sep. 11, 2003

(54) P.F. 30-007 peach tree

(76) Inventor: Paul Jan Friday, Coloma, MI (US)

Correspondence Address: Paul Jan Friday, P.O. Box 850, Coloma, MI, 49038, US

(21) Appl. No.: 10/090,161

(22) Filed: Mar. 5, 2002

(51) Int. Cl. A01H 5/00

(52) U.S. Cl. PLT/199

(57) ABSTRACT

A new and distinct variety of peach, Prunus persica, tree having the following unique combination of desirable features:

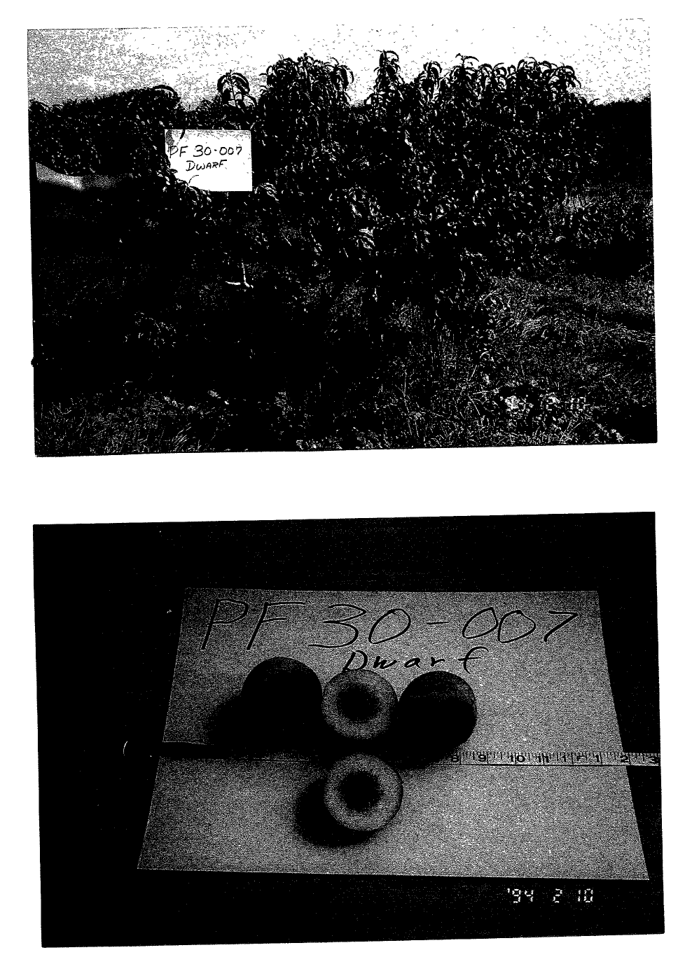

1. The new and distinct variety of peach tree is of “dwarf” spreading growth and a regular and productive bearer of large peaches having a diameter of between 2½″ and 2¾″.

2. Producing a very firm fruit having a resilient flesh texture.

3. Blossoms are partially showy when in full bloom.

4. A substantially spherical fruit with skin of lavender-red color overlying light to whitish yellow at maturity.

5. A very late-season maturing fruit of good taste.

6. A very late-season maturing fruit of good storage and shelf life.

### ORIGIN OF VARIETY

The new peach tree (hereinafter referred to as the P.F. 30-007 peach tree) was originated by Paul Friday in the experimental orchard, which is maintained for the purposes of breeding peach trees, at Paul Friday Farms Inc., located in Coloma, Mich. Coloma is located in the southwest section of Michigan.

In an ongoing mass selection breeding program, superior seedlings of unrecorded parentage are maintained as seed sources for the production of seeds which are collected and planted in mass. The seed producing parent trees are maintained solely as proprietary trees for breeding purposes and have not been released from the experimental orchard, where such trees can be evaluated for their adaptability to local and regional growing conditions. Seeds resulting from open pollination of the trees in the experimental orchard are regularly planted in mass to produce new populations of seedlings which are cultured and monitored to maturity. Trees with superior attributes are retained for further observation and testing, and contribute seeds to advancing generations of new populations of seedlings.

The tree of this application, P.F.30-007, was a selection from one such a seedling population, and was based on the numerous superior genetic attributes of this tree which are described in the botanical description to follow. While not comprehensive, the details of the botanical description to follow are believed to be a reasonably complete botanical description of the tree of this disclosure.

### ASEXUAL REPRODUCTION OF THE VARIETY

The new and distinct variety of peach tree was asexually propagated by budding as performed in the experimental orchard of Paul Friday Farms Inc., located in Coloma, Mich. The asexual propagation demonstrates that such reproduction of the characteristics of the tree are consistent and are established and transmitted through succeeding propagation.

### SUMMARY OF THE VARIETY

The new and very distinct variety of peach tree is a dwarf in stature with spreading growth and a regular and productive bearer of peaches. A distinct characteristic of the P.F. 30-007 peach tree is its short internodes. The blossoms are characterized by being contracted or partially spread when in full bloom. At the same time the petals of the blossoms are of lesser length than the length of petals of the normal showy blossom as exemplified by the Loring peach blossom.

The blossoms of the present peach tree at full bloom may be characterized as being partially non-showy.

The petiole and basal part of the leaf usually have two leaf glands present on each side of the leaf.

The fruit at maturity is large, having flesh of very clear yellow with red around the pit and slight red color into the flesh.

The skin is smooth having moderate down and is of slightly lavender to purple-red color overlying light or whitish yellow. At maturity the peach is spherical having an average diameter ranging between about 2½″ to 3″.

The fruit has a firm flesh and may be described as resilient to the extent that the flesh is yieldable and restorable to its original state when subjected to impact forces, which may cause permanent deformities in peaches of the commercial varieties. The firmness of the fruit facilitates handling and packaging of the peaches without damaging the same for shipment. This results in less spoilage and also increases the shelf life.

The fruit matures in the very late part of the peach growing season in southwestern Michigan. The fruit as mentioned heretofore is of slightly lavender to purple-red color overlying light or whitish yellow and has a very attractive appearance.

### BRIEF DESCRIPTION OF THE PHOTOGRAPHS

In the accompanying photographic illustrations, the top photograph shows a tree in its fifth leaf (i.e., a tree that has been transplanted five seasons since it grew two seasons in the nursery), demonstrating the small size, or “dwarf”, and spreading characteristics of this cultivar.

The bottom photograph shows two whole fruit with one fruit bisected across the axis showing the extent of the red coloration of flesh from the pit well. The otherwise clear yellow flesh is also shown. A tape measure is included to show the good size of this fruit.

### DESCRIPTION OF VARIETY

The detailed botanical description of the foliage and fruit of the new variety of peach tree is based upon observations of the specimens grown at Coloma, Mich. with the color terminology, other than the terminology expressed in common terms, in accordance with the Panatone Matching System (PMS) as used internationally to identify printed colors.

Tree:

Height.—Unpruned 10′.

Width.—Unpruned 12′.

Size.—Small to medium.

Vigor.—Medium.

Density.—Light.

Form.—Spreading.

Production.—Productive — requires thinning.

Bearer.—Consistent.

Disease resistance to bacterial leaf and fruit spot.—Very good.

Trunk:

Bark.—Gray.

Size.—Small to medium.

Surface.—Smooth.

Branches:

Size.—Small to medium.

Surface.—Smooth to medium.

Lenticels.—Substantial number of medium size.

Color.—Gray.

Leaves:

Size.—Average length 6½″ — average width 1⅝″.

Form.—Lanceolate — with elongated point.

Thickness.—Medium.

Texture.—Medium.

Margin.—Finely serrated.

Petiole.—Medium length — medium thickness.

Gland.—Normally 2 glands present on each side of the petiole or basal part of the leaf.

Color.—Upper surface green (553) — lower surface dull green (370).

Flower buds:

Size.—Medium.

Length.—Medium.

Flowers:

Blooming period.—May 3, 2001 to May 6, 2001.

Size.—Medium to large — 1¼″ diameter.

Pollen.—Present, self-fertilizing.

Color.—Pink.

Fruit:

Maturity when described.—Firm ripe.

Date of first picking.—Sep. 12, 2001.

Date of last picking.—Sep. 18, 2001.

Size.—Medium-large — 2½″ to 2¾″.

Form.—Spherical.

Suture.—Bulging — but rounded and smooth.

Skin:

Thickness.—Medium.

Texture.—Medium.

Tendency to crack.—None.

Down.—Moderate.

Color.—Lavender to purple-red (222) — background color light or whitish-yellow (100).

Flesh:

Texture.—Firm, non-melting.

Ripens.—Evenly.

Flavor.—Very good.

Aroma.—Pleasant.

Eating quality.—Excellent.

Color.—Yellow (120) — red around the pit and slightly into flesh (194).

Stone:

Type.—Freestone.

Size.—Small — average length 1⅜″ — average width ⅞″ — average thickness 9/16″.

Form.—Ovid.

Base.—Straight.

Apex.—Pointed.

Sides.—Nearly equal.

Surface.—Furrowed.

Color.—Brown-red (477).

Tendency to crack.—About 2%.

Use: Desert

Shipping quality: Very good.

Keeping quality: Very good.

The tree and its fruit herein described may vary in slight detail as a result of differences in climatic or soil conditions or cull practices under which the tree may be grown. It is to be understood that the description of the new variety as set forth herein is that of the tree grown under the ecological conditions prevailing at Coloma, Mich.

What is claimed is:

1. A new and distinct variety of peach tree substantially as illustrated and described as a compact, “dwarf” tree, a consistent bearer of lavender-red colored, good sized fruit for the very late season, said fruit having firm flesh at maturity.
